(19) United States
(12) Patent Application Publication
(10) Pub. No.: US 2026/0134512 A1
(43) Pub. Date: May 14, 2026

(54) REDUCING THE IMPACT OF VARYING CT CONVOLUTION KERNELS ON MACHINE LEARNING ALGORITHMS

(71) Applicant: ONC.AI, Inc., San Carlos, CA (US)

(72) Inventors: Taly Gilat Schmidt, Whitefish Bay, WI (US); Petr Jordan, Emerald Hills, CA (US)

(21) Appl. No.: 19/388,599

(22) Filed: Nov. 13, 2025

Related U.S. Application Data
(60) Provisional application No. 63/720,284, filed on Nov. 14, 2024.

(51) Int. Cl. G06T 5/50; A61B 6/00; A61B 6/03; A61B 6/50; G06T 5/20; G06T 5/70; G06T 5/73; G06T 7/00; G06T 7/12

(52) U.S. Cl. CPC G06T 5/50; A61B 6/032; A61B 6/505; A61B 6/5258; A61B 6/563; G06T 5/20; G06T 5/70; G06T 5/73; G06T 7/0012; G06T 7/12; G06T 2207/10081; G06T 2207/20081; G06T 2207/20192; G06T 2207/30008; G06T 2207/30061; G06T 2207/30096; G06T 2207/30168

(57) ABSTRACT

A system and method of harmonizing computed tomography (CT) images to reduce variability introduced by differing reconstruction kernels. The method includes acquiring a CT image of a plurality of regions of a patient. The method includes calculating, by a processing device, a noise power spectrum (NPS) of a uniform region among the plurality of regions. The method includes selecting, from a library of reconstruction kernels, a first reconstruction kernel based on the NPS of the uniform region of the plurality of regions. The method includes generating a harmonized CT image based on the first reconstruction kernel and the CT image.

### CROSS-REFERENCE TO RELATED APPLICATIONS

This application claims the benefit of U.S. Provisional Application No. 63/720,284 entitled “REDUCING THE IMPACT OF VARYING CT CONVOLUTION KERNELS ON MACHINE LEARNING ALGORITHMS,” filed Nov. 14, 2025, the disclosure of which is incorporated herein by reference in its entirety.

### TECHNICAL FIELD

The present disclosure relates generally to machine learning, and more particularly, to systems and methods of reducing the impact of varying Computed Tomography (CT) convolution kernels on machine learning algorithms.

### BACKGROUND

The Convolution Kernel setting is a parameter used to reconstruct images from Computed Tomography scanners. The Convolution Kernel represents a filtering operation performed during image reconstruction. Kernels can be selected to balance the sharpness of image features against the amount of image noise. Different kernel settings affect the visual appearance of the image as well as the information stored in the image.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1 is a block diagram depicting an example environment for reducing the impact of varying CT convolution kernels on machine learning algorithms, according to some embodiments;

FIG. 2 illustrates examples of CT images reconstructed using different parameters to demonstrate the variability introduced by reconstruction kernels and nonlinear reconstruction algorithms, according to some embodiments;

FIG. 3A illustrates example noise patterns corresponding to white noise and correlated noise, according to some embodiments;

FIG. 3B illustrates a graph of noise power spectra for white noise and correlated noise, according to some embodiments;

FIG. 4 is a graph illustrating a comparison of noise power spectra (NPS) for multiple CT scanners based on phantom data corresponding to different CT reconstruction kernels, according to some embodiments;

FIG. 5 illustrates an example scan of a uniform object in which multiple ROIs are extracted for noise analysis, according to some embodiments;

FIG. 6 illustrates a flow diagram of an example procedure for reducing the impact of varying CT convolution kernels on machine learning algorithms, according to some embodiments;

FIG. 7 is a flow diagram depicting a method of producing a harmonized CT image, according to some embodiments; and

FIG. 8 is a block diagram of an example computing device 800 that may perform one or more of the operations described herein, in accordance with some embodiments.

### DETAILED DESCRIPTION

As used herein, the terms kernel, convolution kernel, reconstruction kernel, and harmonization filter refer to the same concept, which is a mathematical function applied during the image reconstruction process in computed tomography (CT) to transform raw projection data from a CT scan into a final CT image. The kernel determines how spatial frequencies are weighted during reconstruction, which directly influences image characteristics. For example, smooth kernels emphasize low-frequency components, reducing noise and improving soft tissue visualization, while sharp kernels emphasize high-frequency components, enhancing edge detail but increasing noise. Because the reconstruction kernel controls the balance between sharpness, noise, and contrast, its selection can significantly alter the appearance of the CT image, potentially affecting diagnostic interpretation and downstream applications such as radiomics and machine learning.

Machine learning algorithms, such as those based on radiomic image features or those that learn features using deep learning, may be impacted by the convolution kernel setting. In real-world data, the selected convolution kernel can vary across patients, across imaging sites, and even within a single patient CT dataset. Convolution kernels also differ among manufacturers. This variation can confound imaging features and introduce bias into machine learning algorithms. For example, for radiomic texture features, the difference in features across kernels can exceed the difference in features between the classes of interest. Furthermore, an imbalanced distribution of kernels across the classes of interest can introduce bias that leads to errors when the algorithm is applied to a new patient cohort.

Techniques exist for transforming an image reconstructed with one convolution kernel to resemble an image reconstructed with a different kernel, provided the kernel functions are known. However, the exact shapes of convolution kernels are typically proprietary to each vendor. Other approaches estimate kernel characteristics by analyzing uniform regions within an image dataset. This method involves selecting multiple regions of interest from homogeneous areas and computing the noise power spectrum (NPS), which reflects the kernel's influence on noise texture. A filter for converting an image from one kernel to another can be derived as the square root of the ratio of the target NPS to the original NPS.

Previous work has included scanning uniform phantoms (test devices) on multiple scanners and multiple kernels so that filters can be estimated for kernel harmonization. From these scans, a library of filters can be created for the kernels represented in the phantom data. However, building a comprehensive library that covers all manufacturers and kernels is challenging, particularly as new reconstruction techniques continue to emerge. Thus, there is a long-felt but unsolved need to solve the problems of addressing the challenges of reducing the impact of varying CT convolution kernels on machine learning algorithms.

Aspects of the present disclosure address the above-noted and other deficiencies by providing a Mitigation and Alignment of Tomographic Convolution Kernels (MATCH) system that harmonizes CT images across different reconstruction kernels. The MATCH system leverages noise texture present in the CT image to infer characteristics of the applied reconstruction kernel. Although the kernel type may be known by name, its precise functional impact on the CT image is often not explicitly defined. By analyzing the noise texture, the MATCH system can reverse-engineer the effective behavior of the kernel. This information is then used to design a linear filter that transforms the image to match a desired target appearance, thereby reducing variability in radiomic features attributable to reconstruction differences and enh ancing the consistency and comparability of extracted features across datasets.

The present embodiments provide a method to estimate the convolution kernel from the specific patient image to be transformed. NPS estimation uses multiple regions of interest (ROIs) extracted from uniform regions of the object. In one embodiment, the method segments the aorta, which is expected to have uniform values in most slices, identifies the centroid of the aorta, extracts a region of interest contained within the aorta, and evaluates the uniformity of the region using image processing operations. The NPS of the image is then estimated from the extracted uniform aorta ROIs using established techniques. These steps may also be applied to other regions of the patient that exhibit uniform values in most slices.

Once the NPS is estimated from the CT image, there are two approaches for determining the harmonization filter. In one approach, the filter is calculated as the square root of the ratio of a target NPS (determined a priori) to the NPS estimated within the image. In another approach, the estimated NPS is compared to NPS curves previously determined for specific kernels, which may be obtained through phantom scans. A correlation coefficient can be used to quantify the match between the estimated NPS and the library of NPS functions. Once a similar kernel is identified, the harmonization filter associated with that kernel is applied.

In some embodiments, an alternative approach for estimating kernel shape uses an edge feature in the image to calculate the modulation transfer function. In this approach, filters may be determined using edge features, such as the edge of the aorta, instead of uniform regions.

The present disclosure further includes methods of using the harmonization filter for improving machine learning performance. One method involves harmonizing all images to a uniform kernel type by identifying a target kernel and filtering all input images to match that kernel using the techniques described above. This reduces variability in image appearance and quantitative imaging features due to kernel differences. Harmonization is performed for all training images and for input images during inference.

Another method uses the harmonization filters as a data augmentation step when training a machine learning algorithm. Each training image is filtered multiple times to generate images representing multiple kernels. This augmentation increases the diversity of the training data to reflect real-world variability and reduces training bias based on kernel differences. In this method, input images are not harmonized during inference.

A further method uses the harmonization filters to generate multiple input channels for machine learning algorithms. Images reconstructed using different kernels may contain complementary information beneficial to the learning process. For instance, softer kernels may better depict larger, low-contrast features, whereas sharper kernels enhance edge details. In this method, each input image is harmonized to multiple kernel types during both training and inference, and the resulting harmonized images are provided as separate input channels to the machine learning algorithm.

In an illustrative embodiment, a MATCH system acquires a CT image of a plurality of regions of a patient. The MATCH system calculates a NPS of a uniform region among the plurality of regions. The MATCH system selects, from a library of reconstruction kernels, a first reconstruction kernel based on the NPS of the uniform region of the plurality of regions. The MATCH system generates a harmonized CT image based one the first reconstruction kernel and the CT image.

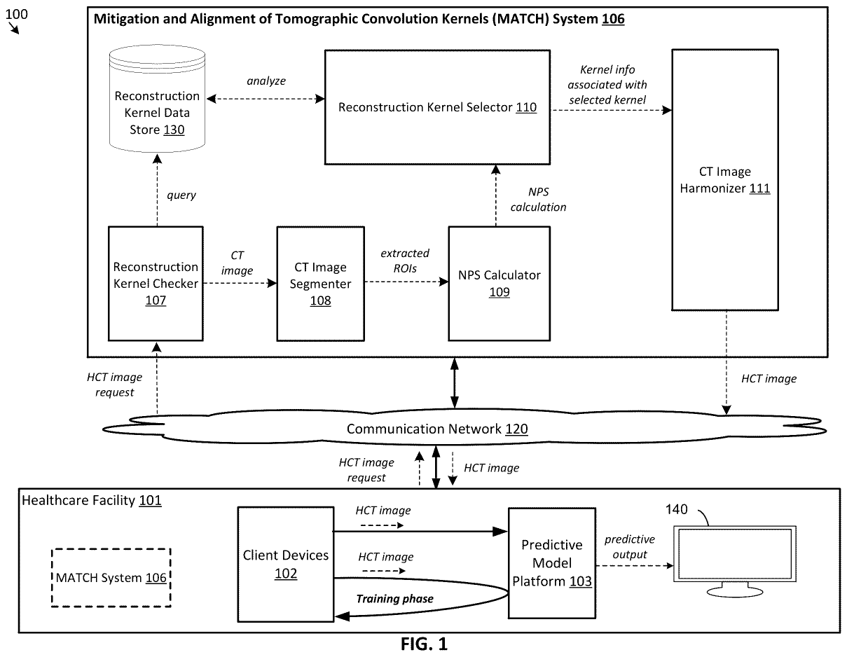

FIG. 1 is a block diagram depicting an example environment reducing the impact of varying CT convolution kernels on machine learning algorithms, according to some embodiments. The environment 100 includes a Mitigation and Alignment of Tomographic Convolution Kernels (MATCH) system 106 and one or more client devices 102 of a healthcare facility 101 that are each communicably coupled together via a communication network 120. The healthcare facility 101 includes a plurality of client devices 102 that are each coupled to a predictive model platform 103, which in turn is coupled to a display 140 for presenting results produced by the predictive model platform 103. A healthcare facility 101 may include hospitals, clinics, imaging centers, medical offices, and outpatient surgical centers.

The MATCH system 106 includes and/or executes a reconstruction kernel checker 107, a CT image segmenter 108, an NPS calculator 109, a reconstruction kernel selector 110, and a CT image harmonizer 111. The MATCH system 106 includes a kernel data store configured to store a library of CT reconstruction kernels. This library includes a plurality of NPS curves that are respectively associated with a plurality of CT reconstruction kernels.

The communication network 120 may be a public network (e.g., the internet), a private network (e.g., a local area network (LAN) or wide area network (WAN), or a combination thereof. In one embodiment, communication network 120 may include a wired or a wireless infrastructure, which may be provided by one or more wireless communications systems, such as Wi-Fi® connectivity to the communication network 120 and/or a wireless carrier system that can be implemented using various data processing equipment, communication towers (e.g., cell towers), etc. The communication network 120 may carry communications (e.g., data, message, packets, frames, etc.) between any other the computing device.

The MATCH system 106, client device 102, and predictive model platform 103 may each be any suitable type of computing device or machine that has a processing device, for example, a server computer (e.g., an application server, a catalog server, a communications server, a computing server, a database server, a file server, a game server, a mail server, a media server, a proxy server, a virtual server, a web server), a desktop computer, a laptop computer, a tablet computer, a mobile device, a smartphone, a set-top box, a graphics processing unit (GPU), etc. In some examples, a computing device may include a single machine or may include multiple interconnected machines (e.g., multiple servers configured in a cluster).

Although FIG. 1 shows only a select number of computing devices (e.g., MATCH system 106, client device 102, etc.), the environment 100 may include any number of computing devices, components, and databases that are interconnected in any arrangement to facilitate the exchange of data between the computing devices.

Still referring to FIG. 1, the reconstruction kernel checker 107 is configured to receive a request (shown in FIG. 1 as HCT image request) to harmonize a CT image, where the request includes a file containing the CT image and corresponding metadata. A CT image may include one or more target lesions. In one embodiment, the terms “target,” “target lesion,” “target subject,” etc. may refer to a nodule, lesion, tumor, metastatic mass or an anatomical structure near (within some defined proximity to) a treatment area. In another embodiment, a target may be a bony structure or bone metastasis. In yet another embodiment a target may refer to soft tissue of a patient. A target may be any defined structure or area capable of being identified and tracked (including the entirety of the patient themselves) as described herein.

Upon receiving the HCT image request, the reconstruction kernel checker 107 may determine whether the metadata indicates the reconstruction kernel used to produce the CT image and, if so, whether the reconstruction kernel data store 130 contains an NPS curve associated with that reconstruction kernel. If the reconstruction kernel for the CT image is known and available in the reconstruction kernel data store 130, the corresponding NPS curve is retrieved and provided to the CT image harmonizer 111, enabling the CT image harmonizer 111 to produce a harmonized CT image based on the selected reconstruction kernel and the CT image.

However, if the reconstruction kernel for the CT image is not known or is not available in the reconstruction kernel data store 130, then the reconstruction kernel checker 107 forwards the CT image to the CT image segmenter 108. The CT image segmenter 108 segments a uniform region (e.g., aorta) of the CT image and extracts one or more regions of interest (ROIs) from the uniform region. The extracted ROIs are then provided to the NPS calculator 109, which calculates an NPS based on the extracted ROIs. The NPS calculator 109 forwards the calculated NPS to the reconstruction kernel selector 110. The reconstruction kernel selector 110 compares the calculated NPS against the reconstruction kernel data store 130 and identifies a particular NPS curve in the library that matches, or is mostly similar to, the calculated NPS. The corresponding reconstruction kernel information is then forwarded to the CT image harmonizer 111, which applies the selected reconstruction kernel to the CT image to produce a harmonized CT image. The CT image harmonizer 111 sends the harmonized CT image to the client device 102.

The client device 102 may include the harmonized CT image in a training dataset used to train the predictive model platform 103 to generate medical predictions (e.g., diagnostic information) about a patient based on their harmonized CT image. Once trained, the predictive model platform 103 may receive additional harmonized CT images from the client device 102, generate predictive outputs based on the CT images, and present the results on a display 140.

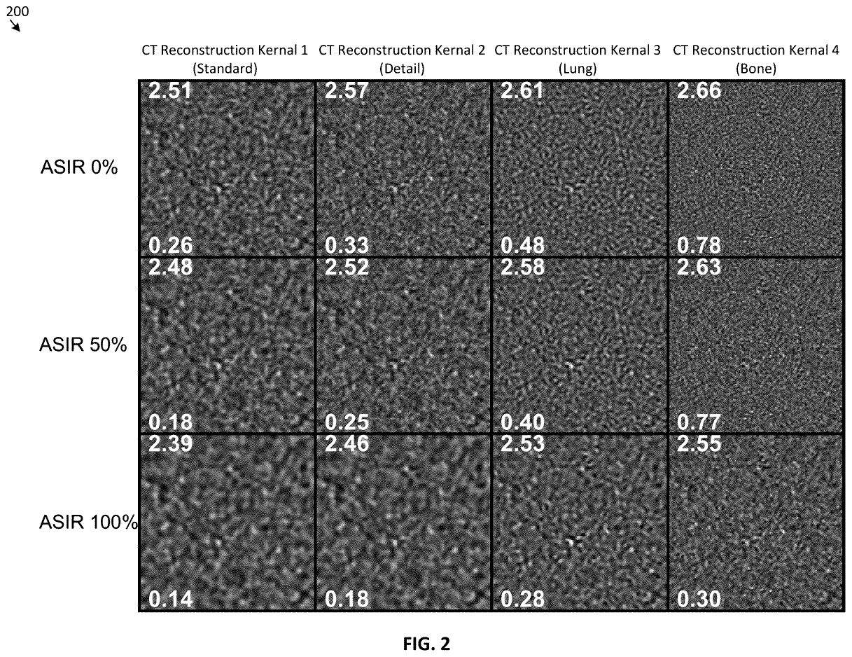

FIG. 2 illustrates examples of CT images reconstructed using different parameters to demonstrate the variability introduced by reconstruction kernels and nonlinear reconstruction algorithms, according to some embodiments. Each column in the figure corresponds to a distinct CT reconstruction kernel, such as Standard, Detail, Lung, or Bone, which significantly influences the texture and appearance of the resulting image. The top row shows images reconstructed with different kernels, and the visual differences in texture across these columns are evident. Each subsequent row represents a different nonlinear reconstruction algorithm, such as Adaptive Statistical Iterative Reconstruction (ASIR), Sinogram Affirmed Iterative Reconstruction (SAFIRE), or deep learning-based methods. Moving down within a single column reveals changes caused by the reconstruction algorithm, which are generally more difficult to harmonize than kernel differences.

The objective is to harmonize images across reconstruction kernels so that images appear more consistent or can be converted from one kernel representation to another, for example, generating an image that appears as if reconstructed with a Lung kernel from an image originally reconstructed with a Standard kernel. While complete harmonization across nonlinear reconstruction algorithms is challenging due to their inherent complexity, the embodiments of the present disclosure can still improve visual consistency and provide acceptable results for practical applications.

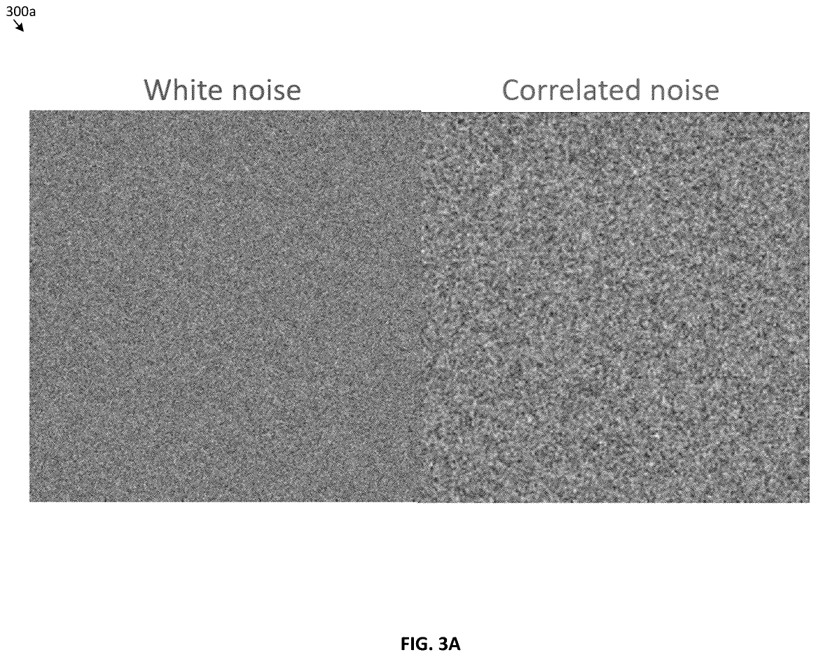

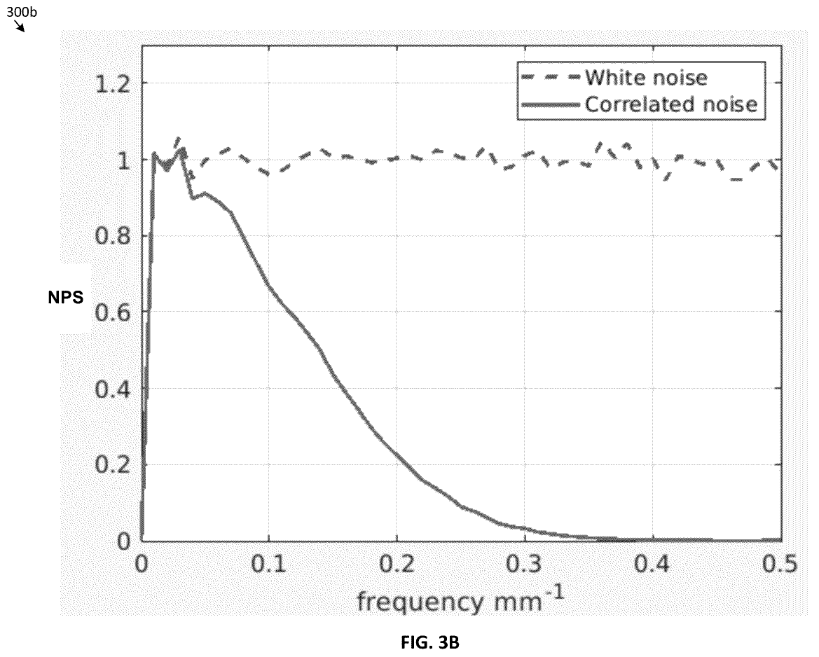

FIG. 3A illustrates example noise patterns corresponding to white noise and correlated noise, according to some embodiments. FIG. 3B illustrates a graph of noise power spectra for white noise and correlated noise, according to some embodiments. The noise power spectrum represents the variance of noise as a function of spatial frequency. White noise exhibits a substantially flat spectrum, indicating that the variance is approximately equal across all spatial frequencies. This characteristic produces a grainy appearance in the image, as shown in FIG. 3A. In contrast, correlated noise exhibits a spectrum in which the variance is greater at lower spatial frequencies and decreases at higher spatial frequencies. This frequency-dependent behavior produces a textured or “blobby” appearance in the image, as shown in FIG. 3A. FIG. 3B further demonstrates this distinction, where the curve corresponding to correlated noise declines with increasing spatial frequency, while the curve corresponding to white noise remains nearly constant. These figures collectively illustrate that CT images typically exhibit correlated noise rather than white noise, and this difference is evident both visually and in the shape of the noise power spectrum.

Understanding the characteristics of the noise power spectrum is important because it directly influences image texture and impacts the ability to harmonize images reconstructed using different algorithms or kernels. White noise, which contributes equally across all spatial frequencies, produces uniform graininess that is relatively straightforward to model. However, CT images generally contain correlated noise, which concentrates variance at lower spatial frequencies and diminishes at higher frequencies. This non-uniform distribution introduces structured patterns that affect perceived image quality and complicate harmonization across reconstruction techniques. By accounting for these frequency-dependent noise properties, reconstruction methods can better preserve diagnostic detail while reducing artifacts associated with correlated noise.

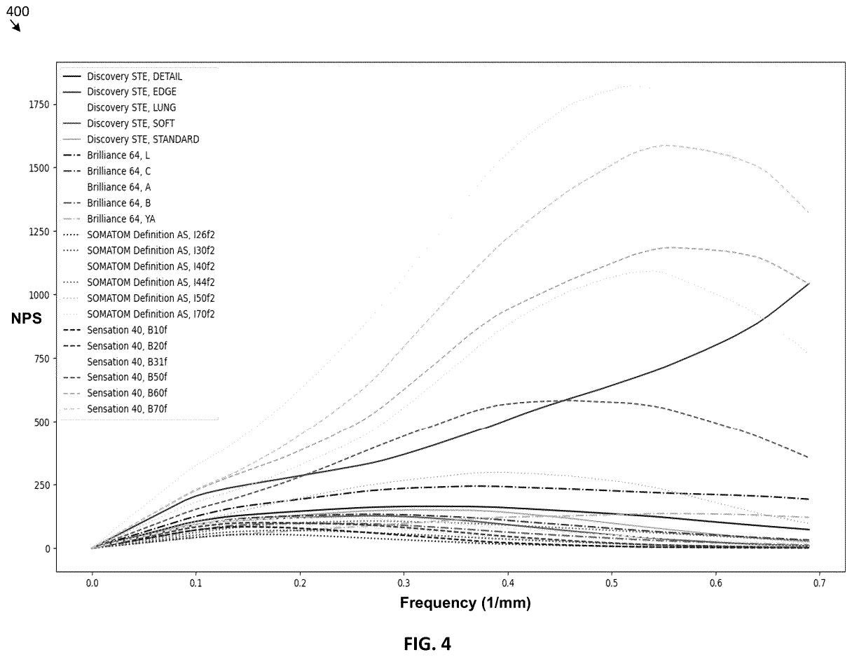

FIG. 4 is a graph illustrating a comparison of noise power spectra (NPS) for multiple CT scanners based on phantom data corresponding to different CT reconstruction kernels, according to some embodiments. Graph 400 plots NPS as a function of spatial frequency for scanners from different manufacturers. Each curve corresponds to a specific kernel configuration within a scanner family. The curves demonstrate that scanners from different manufacturers implement kernels with differing noise profiles, even when the kernels are nominally similar in function. This variability across manufacturers and kernel families highlights the challenge of harmonizing image appearance and noise texture in multi-scanner environments. Graph 400 serves as a library of NPS measurements derived from phantom data across multiple manufacturers, illustrating the diversity of reconstruction behaviors and the importance of accounting for kernel-specific differences when performing image harmonization or quality standardization.

The MATCH system 106 is configured to calculate the correlated noise curve shown in FIG. 3B. The shape of this curve is determined by the reconstruction kernel used to generate the image and serves as a tool for reverse engineering the reconstruction parameters that produced the observed image texture. The NPS can be measured by analyzing multiple regions within a uniform object that contains only noise. By applying a mathematical procedure to these regions, the MATCH system 106 generates the correlated noise curve. The phantom data corresponding to different CT reconstruction kernels, as illustrated in FIG. 4, is used to create a library of noise power spectrum curves across multiple scanners. This library enables the MATCH system 106 to match an input image to a corresponding kernel profile and transform the image so that its noise characteristics approximate those of a different kernel, thereby facilitating harmonization across reconstruction settings.

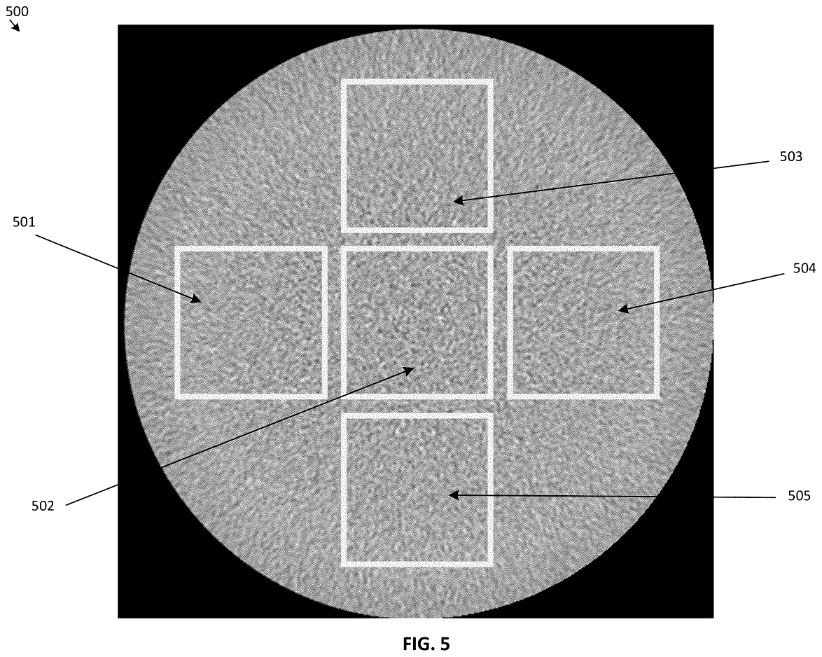

FIG. 5 illustrates an example scan of a uniform object in which multiple ROIs are extracted for noise analysis, according to some embodiments. In the illustrated example, five ROIs (ROI 501-ROI 505) are shown for clarity; however, in practice, the procedure may involve extracting hundreds of ROIs that are spatially separated and distributed across slices. In some embodiments, the MATCH system 106 calculates the in-plane NPS using a procedure that begins by scanning a uniform object and extracting hundreds of ROIs that are separated in space and across slices. Estimating properties of noise involves many measurements, so numerous ROIs are analyzed. For each ROI, the mean value is subtracted to isolate the noise component. A two-dimensional Fourier transform is then applied to each ROI, and the resulting transforms are averaged to obtain a composite representation of noise characteristics. Because the NPS is typically assumed to be radially symmetric, the averaged data is further processed by radial averaging to produce a one-dimensional curve representing the NPS. This curve reflects the variance of noise as a function of spatial frequency and is influenced by the reconstruction kernel used to generate the image. By calculating the NPS in this manner, the MATCH system 106 obtains a representation of noise characteristics that can be used to identify reconstruction parameters and harmonize images across different kernels.

The present disclosure provides methods for harmonizing CT images across different reconstruction kernels to reduce variability and bias in machine learning algorithms. Variations in reconstruction kernels can introduce systematic differences in image appearance, such as edge sharpness, noise magnitude, and noise texture. These differences can lead to biases in machine learning models, as images from different sites or scanners may exhibit inconsistent characteristics unrelated to pathology. To address this, the disclosed methods aim to control for kernel-induced variation so that radiomic features and machine learning predictions are not confounded by reconstruction settings.

In one embodiment, harmonization is achieved by generating filters that transform an image reconstructed with an original kernel to approximate the appearance of an image reconstructed with a target kernel. This process assumes that the kernel type and reconstruction algorithm for each dataset are known and that access to scanners is available for calibration. A uniform phantom is scanned using each scanner and reconstruction kernel to produce reference data. From these scans, tables of noise power spectra are created for each setting, characterizing the distribution of noise energy across spatial frequencies.

Using these NPS tables, a library of harmonization filters is developed. For each frequency component, the ratio of the target NPS to the original NPS is computed, and the square root of this ratio is taken to account for the squared nature of noise power. The resulting function defines a harmonization filter in the frequency domain. Because raw ratios can be noisy, the filters are typically fitted to smooth functions, such as polynomials or cubic splines, to improve stability and generalizability. These fitted filters are stored in a library indexed by kernel type and scanner configuration.

When the MATCH system 106 receives a CT image, it determines the kernel associated with the image and identifies the desired target kernel. The MATCH system 106 then retrieves the appropriate harmonization filter from the library, which was derived from phantom data collected across multiple scanners and manufacturers. The filter is applied to the image in the frequency domain, typically by performing two-dimensional filtering on each slice. This operation adjusts the noise texture and related characteristics of the image to match the target kernel, thereby reducing variability in radiomic features attributable to reconstruction differences and improving consistency across datasets.

In some cases, the MATCH system 106 may receive a CT image without kernel metadata or with a kernel that is not represented in the existing harmonization filter library. This creates a challenge because harmonization typically relies on knowing the kernel type and applying a corresponding filter derived from phantom-based measurements. To address this, the present disclosure provides a practical approach for harmonizing images when kernel information is missing or incomplete.

The process begins by creating an initial library of harmonization filters using datasets with known kernels and reconstruction algorithms. A uniform phantom is scanned across multiple scanners and reconstruction settings to generate reference data. From these scans, tables of noise power spectra are computed for each kernel and reconstruction configuration. Each harmonization filter is calculated as the square root of the ratio of the target NPS to the original NPS for each spatial frequency component, and the resulting filters are typically fitted to smooth functions, such as polynomials or cubic splines, to reduce noise and improve stability. These fitted filters are stored in a library indexed by kernel and reconstruction type.

For a new dataset, if the kernel and reconstruction algorithm are known, the MATCH system 106 retrieves the appropriate filter from the library and applies it to the image, typically by performing two-dimensional filtering on each slice. If the kernel or reconstruction algorithm is unknown, or if the kernel is not represented in the library, the MATCH system 106 estimates the NPS directly from the image data. To do this, the MATCH system 106 identifies a region of the image that is relatively uniform, such as the aorta, and extracts multiple ROIs from this area. The NPS is computed from these ROIs and compared to the NPS profiles in the library to determine which known kernel the image most closely resembles. The MATCH system 106 then selects the corresponding filter and applies it to harmonize the image. This approach enables harmonization even when kernel metadata is missing or when the kernel is not included in the precomputed library, thereby reducing variability and bias in machine learning algorithms caused by differences in reconstruction settings.

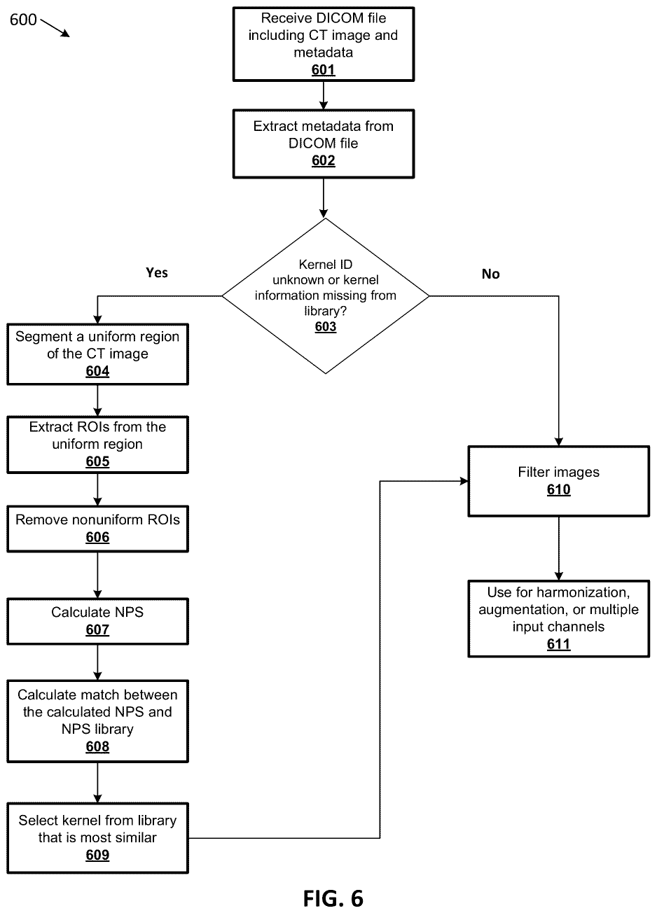

FIG. 6 illustrates a flow diagram of an example procedure for reducing the impact of varying CT convolution kernels on machine learning algorithms, according to some embodiments. Although the operations are depicted in FIG. 6 as integral operations in a particular order for purposes of illustration, in other implementations, one or more operations, or portions thereof, are performed in a different order, or overlapping in time, in series or parallel, or are omitted, or one or more additional operations are added, or the method is changed in some combination of ways. In some embodiments, the procedure 600 may be performed by processing logic that includes hardware (e.g., circuitry, dedicated logic, programmable logic, microcode, etc.), firmware, or a combination thereof. In some embodiments, some or all operations of procedure 600 may be performed by one or more components (e.g., reconstruction kernel checker 107, CT image segmenter 108, NPS calculator 109, reconstruction kernel selector 110, CT image harmonizer 111) of the MATCH system 106 in FIG. 1.

At operation 601, the MATCH system 106 receives a Digital Imaging and Communications in Medicine (DICOM) file containing a CT image of a patient, along with associated metadata. This metadata may include patient information (e.g., age, weight, gender), scan parameters (e.g., exposure time, rotation time, slice thickness), a reconstruction kernel identifier (ID), and the acquisition date. In some embodiments, the reconstruction kernel ID may be missing from the metadata. The reconstruction kernel is a mathematical filter applied during the image reconstruction process that shapes how raw scan data is converted into visual images. As discussed herein, the reconstruction kernel can significantly influence the appearance, sharpness, noise, and contrast of the final CT image.

At operation 602, the match system 106 extracts the metadata from the dicome file.

At operation 603, the MATCH system 106 determines whether the reconstruction kernel ID is unknown or whether reconstruction kernel information is missing from the kernel library. If the metadata is missing the reconstruction kernel ID, the MATCH system 106 proceeds to operation 604. Similarly, if the metadata includes a reconstruction kernel ID but there is no performance information about that particular reconstruction kernel in the kernel library, the MATCH system 106 also proceeds to operation 604. Otherwise, the MATCH system 106 proceeds to operation 610.

At operation 604, the MATCH system 106 segment a uniform region (e.g., aorta) depicted in the CT image.

At operation 605, the MATCH system 106 extracts ROIs from the uniform region.

At operation 606, the MATCH system 106 removes any nonuniform ROIs.

At operation 607, the MATCH system 106 calculates an NPS based on the extracts ROIs.

At operation 608, the MATCH system 106 compares the calculated NPS against a library of reconstruction kernels. This library includes a plurality of NPS curves that are respectively associated with a plurality of reconstruction kernels (sometimes referred to as harmonization filters). Based on this comparison, the MATCH system 106 identifies a particular NPS curve contained in the library that matches (or is most similar to) the calculated NPS.

At operation 609, the MATCH system 106 selects the reconstruction kernel from the library that is associated with the particular NPS curve.

At operation 610, the MATCH system 106 applies the selected reconstruction kernel to the CT image to produce a harmonized CT image.

At operation 611, the MATCH system 106 uses the harmonized CT image any number of applications, such as harmonization, augmentation, or multiple input channels.

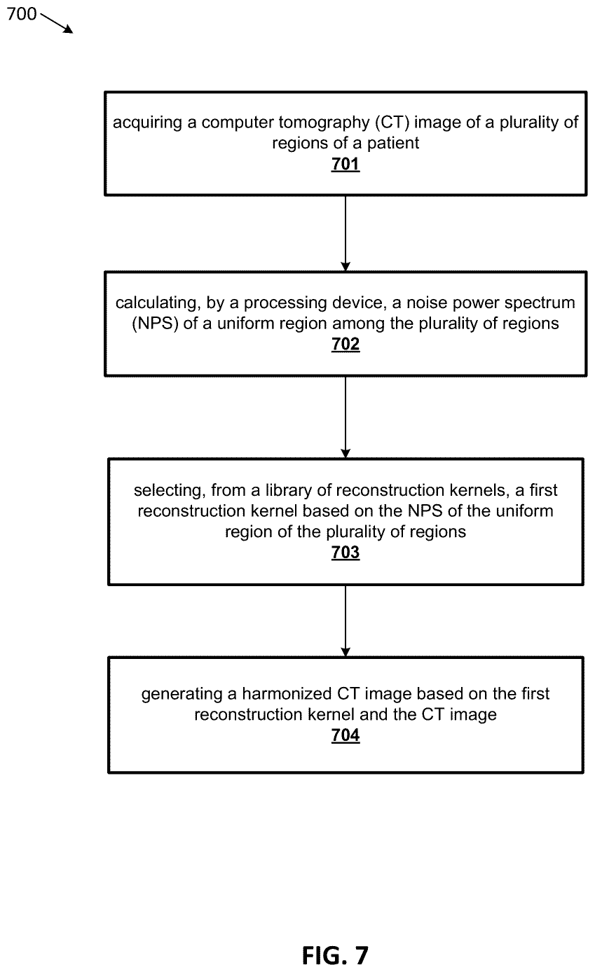

FIG. 7 is a flow diagram depicting a method of producing a harmonized CT image, according to some embodiments. Method 700 may be performed by processing logic that may include hardware (e.g., circuitry, dedicated logic, programmable logic, a processor, a processing device, a central processing unit (CPU), a system-on-chip (SoC), etc.), software (e.g., instructions and/or an application that is running/executing on a processing device), firmware (e.g., microcode), or a combination thereof. In some embodiments, method 700 may be performed the MATCH system 106 in FIG. 1.

With reference to FIG. 7, method 700 illustrates example functions used by various embodiments. Although specific function blocks (“blocks”) are disclosed in method 700, such blocks are examples. That is, embodiments are well suited to performing various other blocks or variations of the blocks recited in method 700. It is appreciated that the blocks in method 700 may be performed in an order different than presented, and that not all of the blocks in method 700 may be performed.

As shown in FIG. 7, the method 700 includes the block 702 of acquiring a computer tomography (CT) image of a plurality of regions of a patient. The method 700 includes the block 704 of calculating, by a processing device, a noise power spectrum (NPS) of a uniform region among the plurality of regions. The method 700 includes the block 706 of selecting, from a library of reconstruction kernels, a first reconstruction kernel based on the NPS of the uniform region of the plurality of regions. The method 700 includes the block 708 of generating a harmonized CT image based on the first reconstruction kernel and the CT image.

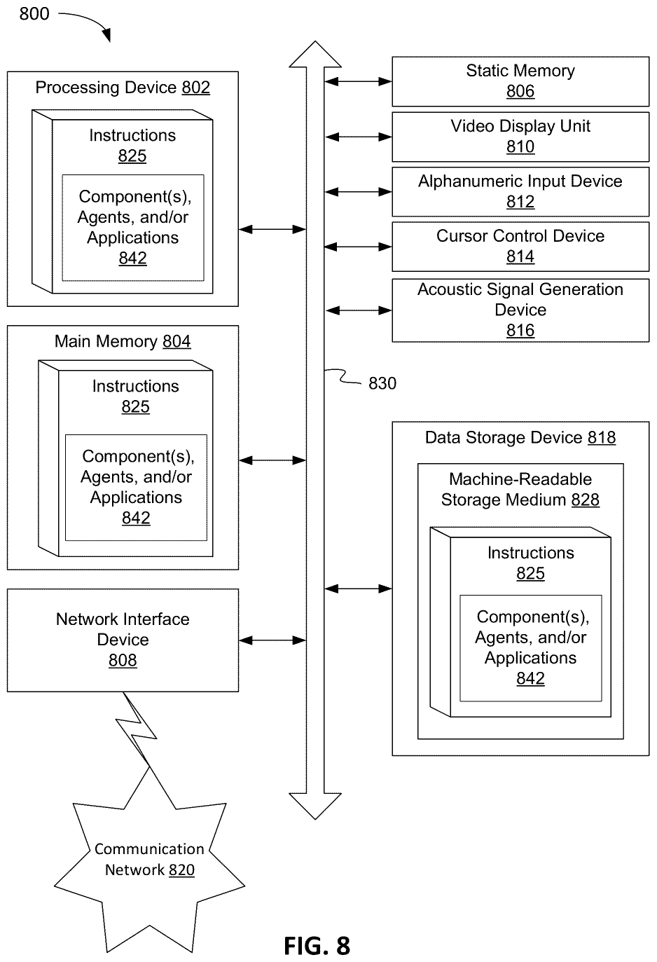

FIG. 8 is a block diagram of an example computing device 800 that may perform one or more of the operations described herein, in accordance with some embodiments. Computing device 800 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 800 may include a processing device (e.g., a general-purpose processor, a PLD, etc.) 802, a main memory 804 (e.g., synchronous dynamic random-access memory (DRAM), read-only memory (ROM)), a static memory 806 (e.g., flash memory and a data storage device 818), which may communicate with each other via a bus 830.

Processing device 802 may be provided by one or more general-purpose processing devices such as a microprocessor, central processing unit, or the like. In an illustrative example, processing device 802 may include a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 802 may also include one or more special-purpose processing devices such as an application specific integrated circuit (ASIC), a field programmable gate array (FPGA), a digital signal processor (DSP), network processor, or the like. The processing device 802 may be configured to execute the operations described herein, in accordance with one or more aspects of the present disclosure, for performing the operations and steps discussed herein.

Computing device 800 may further include a network interface device 808 which may communicate with a communication network 820. The computing device 800 also may include a video display unit 810 (e.g., a liquid crystal display (LCD) or a cathode ray tube (CRT)), an alphanumeric input device 812 (e.g., a keyboard), a cursor control device 814 (e.g., a mouse) and an acoustic signal generation device 816 (e.g., a speaker). In one embodiment, video display unit 810, alphanumeric input device 812, and cursor control device 814 may be combined into a single component or device (e.g., an LCD touch screen).

Data storage device 818 may include a computer-readable storage medium 828 on which may be stored one or more sets of instructions 825 that may include instructions for one or more components, agents, and/or applications 842 (e.g., reconstruction kernel checker 107, CT image segmenter 108, NPS calculator 109, reconstruction kernel selector 110, CT image harmonizer 111 in FIG. 1) for carrying out the operations described herein, in accordance with one or more aspects of the present disclosure. Instructions 825 may also reside, completely or at least partially, within main memory 804 and/or within processing device 802 during execution thereof by computing device 800, main memory 804 and processing device 802 also constituting computer-readable media. The instructions 825 may further be transmitted or received over a communication network 820 via network interface device 808.

While computer-readable storage medium 828 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

Unless specifically stated otherwise, terms such as “acquiring,” “calculating,” “selecting,” “generating,” “training,” “determining,” “identifying,” “extracting,” “assessing,” “receiving,” “transmitting,” or the like, refer to actions and processes performed or implemented by computing devices that manipulates and transforms data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein are meant as labels to distinguish among different elements and may not necessarily have an ordinal meaning according to their numerical designation.

Examples described herein also relate to an apparatus for performing the operations described herein. This apparatus may be specially constructed for the required purposes, or it may include a general-purpose computing device selectively programmed by a computer program stored in the computing device. Such a computer program may be stored in a computer-readable non-transitory storage medium.

The methods and illustrative examples described herein are not inherently related to any particular computer or other apparatus. Various general-purpose systems may be used in accordance with the teachings described herein, or it may prove convenient to construct more specialized apparatus to perform the required method steps. The required structure for a variety of these systems will appear as set forth in the description above.

The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. § 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

The foregoing description, for the purpose of explanation, has been described with reference to specific embodiments. However, the illustrative discussions above are not intended to be exhaustive or to limit the present disclosure to the precise forms disclosed. Many modifications and variations are possible in view of the above teachings. The embodiments were chosen and described to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the present disclosure is not to be limited to the details given herein, but may be modified within the scope and equivalents of the appended claims.

What is claimed is:

1. A method comprising:
acquiring a computer tomography (CT) image of a plurality of regions of a patient;
calculating, by a processing device, a noise power spectrum (NPS) of a uniform region among the plurality of regions;
selecting, from a library of reconstruction kernels, a first reconstruction kernel based on the NPS of the uniform region of the plurality of regions; and
generating a harmonized CT image based on the first reconstruction kernel and the CT image.

2. The method of claim 1, further comprising:
training, based on the harmonized CT image, a machine learning model to predict medical information about the patient.

3. The method of claim 1, wherein the library of reconstruction kernels comprises a plurality of NPS curves respectively associated with a plurality of reconstruction kernels.

4. The method of claim 3, wherein selecting the first reconstruction kernel based on the NPS of the uniform region of the plurality of regions further comprises:
selecting, from the library of reconstruction kernels, a first NPS curve from the plurality of NPS curves by determining that the first NPS curve is a closest match to the NPS of the uniform region; and
determining that the first reconstruction kernel is associated with the first NPS curve.

5. The method of claim 1, wherein selecting the first reconstruction kernel based on the NPS of the uniform region further comprises:
calculating a square root of a ratio of a target NPS to the NPS of the uniform region.

6. The method of claim 1, wherein the CT image is contained in a file, and further comprising:
determining an absence of metadata indicating a distinct reconstruction kernel used to generate the CT image; and
wherein calculating the NPS of the uniform region of the plurality of regions is performed in response to determining the absence of the metadata.

7. The method of claim 1, wherein the CT image is contained in a file indicating a distinct reconstruction kernel used to generate the CT image, and further comprising:
determining, from among the library of reconstruction kernels, an absence of an NPS curve associated with the distinct reconstruction kernel; and
wherein calculating the NPS of the uniform region of the plurality of regions is performed in response to determining the absence of the NPS curve associated with the distinct reconstruction kernel.

8. The method of claim 1, wherein calculating the NPS of the uniform region among the plurality of regions further comprises:
identifying a centroid of the uniform region;
extracting, using the centroid, a region of interest from the uniform region to produce an extracted region; and
assessing a uniformity of the extracted region based on image characteristics.

9. The method of claim 1, further comprising:
selecting, from the library of reconstruction kernels, the first reconstruction kernel based on one or more edge features in the CT image.

10. The method of claim 1, wherein the processing device resides in a cloud environment, and further comprising:
receiving, by the processing device from a healthcare provider, a request to harmonize the CT image of the patient; and
transmitting, by the processing device, the harmonized CT image to the healthcare provider.

11. The method of claim 1, further comprising:
inputting the harmonized CT image into a machine learning model to generate an output indicative of one or more clinical, anatomical, or prognostic characteristics of the patient.

12. A system comprising:
a memory; and
a processing device, operatively coupled to the memory, to:
acquire a computer tomography (CT) image of a plurality of regions of a patient;
calculate, by a processing device, a noise power spectrum (NPS) of a uniform region among the plurality of regions;
select, from a library of reconstruction kernels, a first reconstruction kernel based on the NPS of the uniform region of the plurality of regions; and
generate a harmonized CT image based on the first reconstruction kernel and the CT image.

13. The system of claim 12, wherein the processing device is further to:
train, based on the harmonized CT image, a machine learning model to predict medical information about the patient.

14. The system of claim 12, wherein the library of reconstruction kernels comprises a plurality of NPS curves respectively associated with a plurality of reconstruction kernels.

15. The system of claim 14, wherein to select the first reconstruction kernel based on the NPS of the uniform region of the plurality of regions, the processing device is further to:
select, from the library of reconstruction kernels, a first NPS curve from the plurality of NPS curves by determining that the first NPS curve is a closest match to the NPS of the uniform region; and
determine that the first reconstruction kernel is associated with the first NPS curve.

16. The system of claim 12, wherein to select the first reconstruction kernel based on the NPS of the uniform region, the processing device is further to:
calculate a square root of a ratio of a target NPS to the NPS of the uniform region.

17. The system of claim 12, wherein the CT image is contained in a file, and wherein the processing device is further to:
determine an absence of metadata indicating a distinct reconstruction kernel used to generate the CT image; and
wherein calculating the NPS of the uniform region of the plurality of regions is performed in response to determining the absence of the metadata.

18. The system of claim 12, wherein the CT image is contained in a file indicating a distinct reconstruction kernel used to generate the CT image, and wherein the processing device is further to:
determine, from among the library of reconstruction kernels, an absence of an NPS curve associated with the distinct reconstruction kernel; and
wherein calculating the NPS of the uniform region of the plurality of regions is performed in response to determining the absence of the NPS curve associated with the distinct reconstruction kernel.

19. The system of claim 12, wherein to calculate the NPS of the uniform region among the plurality of regions, the processing device is further to:
identify a centroid of the uniform region;
extract, using the centroid, a region of interest from the uniform region to produce an extracted region; and
assess a uniformity of the extracted region based on image characteristics.

20. The system of claim 12, wherein the processing device is further to:
select, from the library of reconstruction kernels, the first reconstruction kernel based on one or more edge features in the CT image.

21. A non-transitory computer-readable medium storing instructions that, when executed by a processing device, cause the processing device to:
acquire a computer tomography (CT) image of a plurality of regions of a patient;
calculate, by the processing device, a noise power spectrum (NPS) of a uniform region among the plurality of regions;
select, from a library of reconstruction kernels, a first reconstruction kernel based on the NPS of the uniform region of the plurality of regions; and
generate a harmonized CT image based on the first reconstruction kernel and the CT image.
